(19) United States
(12) Patent Application Publication
(10) Pub. No.: US 2025/0009257 A1
(43) Pub. Date: Jan. 9, 2025

(54) ANTHROPOMETRIC GROWTH APPARATUS

(71) Applicant: STELLENBOSCH UNIVERSITY, Stellenbosch (ZA)

(72) Inventors: Klara McCLUNAN, Stellenbosch (ZA); Evette VAN NIEKERK, Stellenbosch (ZA)

(73) Assignee: STELLENBOSCH UNIVERSITY, Stellenbosch, WC (ZA)

(21) Appl. No.: 18/710,990

(22) PCT Filed: Nov. 17, 2022
(86) PCT No.: PCT/IB2022/061075

§ 371 Date: May 16, 2024

(30) Foreign Application Priority Data
Nov. 17, 2021 (ZA) 2021/09135

(51) Int. Cl. A61B 5/107

(52) U.S. Cl. CPC A61B 5/1072; A61B 2503/045

(57) ABSTRACT

An anthropometric growth apparatus (101, 201, 501) for measuring the anthropometry of an infant is provided. The apparatus comprises a base (103, 203, 503) with a digital scale assembly (105, 205, 505) mounted to the base and configured to measure a weight of the infant. A digital length measurement assembly (107, 207, 507) is mounted to the base and configured to measure a length of the infant. A head circumference measurement assembly (109, 209, 509) is mounted to the base and includes an array of distance sensors (111, 211, 511) provided on a support (113, 213, 513) configured to position the array of distance sensors at a selected distance from the head of the infant for non-invasive measurement of the head circumference in use. The apparatus may further be used for body composition and gestational age determination.

### CROSS-REFERENCE(S) TO RELATED APPLICATIONS

This application claims priority from South African provisional patent application number 2021/09135 filed on 17 Nov. 2021, which is incorporated by reference herein.

### FIELD OF THE INVENTION

This invention relates to an anthropometric growth apparatus. In particular, it relates to an anthropometric growth apparatus for obtaining anthropometric measurements such as weight, length and head circumference of human subjects, more particularly infants, in a minimally invasive manner.

### BACKGROUND TO THE INVENTION

The anthropometry, such as weight, length, head circumference, and foot length of new-born infants, including preterm infants, is often not measured accurately, or recorded in patient folders correctly. Sometimes measurements cannot be taken by medical personnel due to the infant being connected to medical equipment such as nasal continuous positive airway pressure (nCPAP) equipment whilst being cared for in a neonatal intensive care unit (NICU). Effective growth monitoring and nutritional and clinical prescriptions require the correct anthropometric measurements to assess, treat and manage preterm, diseased, or otherwise fragile infants appropriately, thereby improving their quality of life and decreasing the length of their hospital stay.

Existing infant measurement instruments may be cumbersome to use requiring the presence of more than one nursing staff member to measure and handle the infant. Many apparatuses only measure one or two anthropometric measurements at a time. Measurement procedures may then be complex and time consuming. Some apparatuses are invasive requiring a lot of manipulation of the preterm or otherwise fragile infant or may require the removal of the infant from their open or closed incubator, increasing the risk of infection to the infant.

Most preterm infants or new-born infants with respiratory distress require nCPAP treatment, at least for the first few days after birth. The nCPAP fixation device or mask employed to administer the therapy restricts access to the infant's head and, accordingly, head circumference measurements may not be taken at all or may be inaccurate.

Specialised and expensive equipment for obtaining anthropometric measurements at the critical early stage of care may not be available in a resource-limited setting.

Accordingly, there is a need for an anthropometric measurement apparatus and system that alleviates the abovementioned problems, at least to an extent.

The preceding discussion of the background to the invention is intended only to facilitate an understanding of the present invention. It should be appreciated that the discussion is not an acknowledgment or admission that any of the material referred to was part of the common general knowledge in the art as at the priority date of the application.

### SUMMARY OF THE INVENTION

In accordance with an aspect of the invention there is provided an anthropometric growth apparatus for measuring the anthropometry of an infant comprising:
a base;
a digital scale assembly mounted to the base and configured to measure a weight of the infant;
a digital length measurement assembly mounted to the base and configured to measure a length of the infant; and
a head circumference measurement assembly mounted to the base which includes an array of distance sensors provided on a support configured to position the array of distance sensors at a selected distance from a head of the infant for non-invasive measurement of a head circumference of the infant in use.

The support may extend partially about the circumference of a head of the infant in use. The distance sensors may be light emitting diode (LED) time-of-flight sensors.

The support for the array of distance sensors may be moveable from a stowed condition in which it is at least partially received within a complementarily shaped receiving formation defined in the base and an operative condition in which it extends transversely from the base so as to be operatively above and extend at least partially around the infant's head in use. The support may include at least one curved arm with distance sensors equally circumferentially spaced along the curved arm. The support may be pivotally attached to the base so as to be collapsible against the base.

The digital length measurement assembly may include a reference board attached at or near an end of the base and extending transversely therefrom in use and a distance sensor at or near the opposite end of the base to the reference board, the distance sensor being configured to measure the distance of the infant, or a target placed at or adjacent the infant, from the distance sensor when the infant is placed against the reference board, for use in calculating a length of the infant.

The digital length measurement assembly may be moveable between a stowed condition in which the digital length measurement assembly is collapsed within or against the base and an operative condition in which the assembly extends transversely from the base and is arranged to measure the length of the infant in use. The reference board and distance sensor of the digital length measurement assembly may be pivotally attached to the base so as to be moveable between the stowed condition the operative condition. The base may have recesses defined therein and configured to receive the reference board and distance sensor in the stowed condition. The distance sensor may be an LED time-of-flight sensor.

The digital scale assembly may include a flat, weighing platform on which the infant is placed for weight measurement in use with a weight sensor on the platform.

The anthropometric growth apparatus may further include a digital foot length measurement assembly.

The anthropometric growth apparatus may include a processing module in communication with at least the digital weight, length and head circumference measurement assemblies to receive signals from the respective assemblies and process the signals to determine the weight, length and head circumference of the infant. The anthropometric growth apparatus may include a display module on the base that is in communication with the processing module and configured to display the measured weight, length and head circumference of the infant in use. The display module may be a touchscreen in some embodiments. The processing module may be configured to transmit the weight, length and heads circumference measurements to a processing module of an external computing device for further processing.

Embodiments of the invention will now be described, by way of example only, with reference to the accompanying drawings.

### BRIEF DESCRIPTION OF THE DRAWINGS

In the drawings:

FIG. 1 is a top perspective view of a first embodiment of an anthropometric growth apparatus for measuring an infant in an operative condition;

FIG. 2 is a top view of the embodiment of FIG. 1 in the operative condition;

FIG. 3 is a side view of the embodiment of FIG. 1 in the operative condition;

FIG. 4 is a top perspective view of the head circumference measurement assembly of the embodiment of FIG. 1;

FIG. 5 is a side view of the head circumference measurement assembly of the embodiment of FIG. 1;

FIG. 6 is a top perspective view of the head circumference measurement assembly of the embodiment of FIG. 1 with a distance sensor module and the ball bearing and magnet on the end of a rotatable shaft shown in exploded, window views;

FIG. 7 is a top perspective view of a second embodiment of an anthropometric growth apparatus for measuring an infant in an operative condition;

FIG. 8 is a top view of the embodiment of FIG. 7 in the operative condition;

FIG. 9 is a front view of the embodiment of FIG. 7 in the operative condition;

FIG. 10 is a side view of the embodiment of FIG. 7 in the operative condition;

FIG. 11 is a top perspective view of the embodiment of FIG. 7 in a disassembled condition;

FIG. 12 is an illustration of a curved support with distance sensors arranged thereon for the measurement and calculation of a head circumference;

FIG. 13 is an illustration of a rectangular-shaped support with distance sensors arranged thereon for the measurement and calculation of a head circumference;

FIG. 14 is a top perspective view of a third embodiment of an anthropometric growth apparatus for measuring an infant in an operative condition;

FIG. 15 is a top perspective view of the embodiment of FIG. 14 in a stowed condition;

FIG. 16 is a top perspective view of the embodiment of FIG. 14 in use to measure a weight of an infant;

FIG. 17 is a top perspective view of the embodiment of FIG. 14 in the operative condition and in use to measure a weight, length and head circumference of an infant; and

FIG. 18 is a top perspective view of the embodiment of FIG. 14 in use with a target placed at the feet of an infant to measure a length of the infant.

### DETAILED DESCRIPTION WITH REFERENCE TO THE DRAWINGS

A multi-parameter anthropometric growth apparatus or device is provided for measuring the anthropometry of children, and more particularly, infants. The measuring apparatus is minimally invasive and may be used on preterm infants or otherwise fragile infants or children, particularly in a limited-resource hospital setting. The multifunctional apparatus conducts key measurements, including at least the weight, length and head circumference of an infant, which are fundamental in monitoring the growth and development of the infant. Gestational age measurement means may also be included in the multifunctional apparatus to further track the growth trends or patterns of the infant. Most, and preferably all, of the measurements take place electronically or digitally, which further enables automatic and accurate recording thereof. When correct daily and/or weekly measurements of these growth parameters are done while an infant is cared for in hospital, the correct clinical and nutritional prescriptions will be given, so that the infant's condition improves, and the length of the hospital stay is reduced. Accordingly, the apparatus optimises infant monitoring and care.

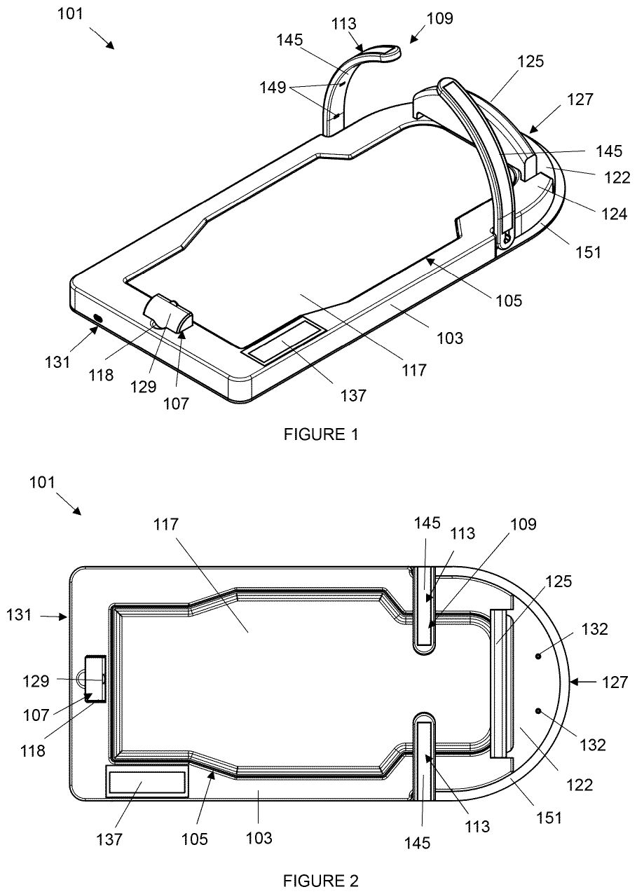

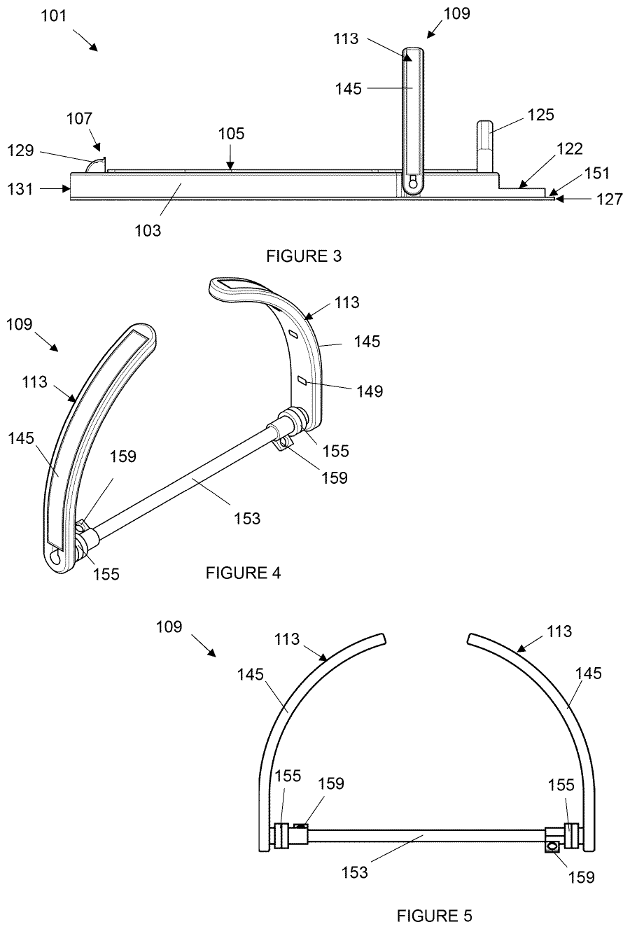

A first embodiment of an anthropometric growth apparatus (101) for measuring an infant is shown in FIGS. 1 to 6. The apparatus (101) comprises a base (103) with a digital scale assembly (105) mounted to the base (103) which is configured to measure a weight of the infant. The digital scale assembly includes a flat and rigid weighing platform (117) on which the infant is placed for weight measurement in use and to which a digital weight sensor is connected.

A digital length measurement assembly (107) configured to measure a length of the infant is also mounted to the base (103). In this embodiment, the digital length measurement assembly (107) comprises a distance sensor (129) mounted to the base (103) near a first end (131) of the base (103). The distance sensor (129) is arranged to measure a distance of the infant from the sensor when the infant is placed on the weighing platform (117) and against a reference board (125). The reference board (125) extends transversely or generally perpendicularly from the base (103) near the opposite end (127) of the base (103) to the end (131) with the distance sensor (129). It will be appreciated that, for the length measurement, the infant may be placed on the apparatus with either his/her head or feet against the reference board. Preferably, the head or crown is placed against the reference board (125) so that the head is near the head circumference measurement assembly described in more detail below. The distance measurement obtained using the distance sensor (129) is used to calculate the length of the infant by subtracting the distance the infant's head or feet is from the sensor from a fixed distance between the distance sensor (129) and the reference board (125). In this embodiment, the distance sensor (129) is a light emitting diode (LED) time-of-flight sensor.

Both the distance sensor (129) and the reference board (125) are pivotally connected to the base (103) so as to be moveable or pivotable between an operative condition and an inoperative condition. In the operative condition, the sensor (129) and reference board (125) extend transversely from the base and are suitably positioned for length measurements of an infant to be carried out with the apparatus. In the stowed condition, the distance sensor (129) and the reference board (125) are collapsed against the base (103) and received within complementarily shaped receiving formations for each defined in the base (103). The distance sensor (129) pivots until it is received into a recess (118) for it defined in the base (103) so as to be flush with the base (103). The reference board (125) is hingedly connected to the base and when collapsed into a complementarily shaped recess (122) defined in the base, it lies flush with the operatively upper surface (124) of the base. The reference board (125) is held in place in the inoperative or stowed condition via fasteners such as clips (132) or magnets provided on the base (103).

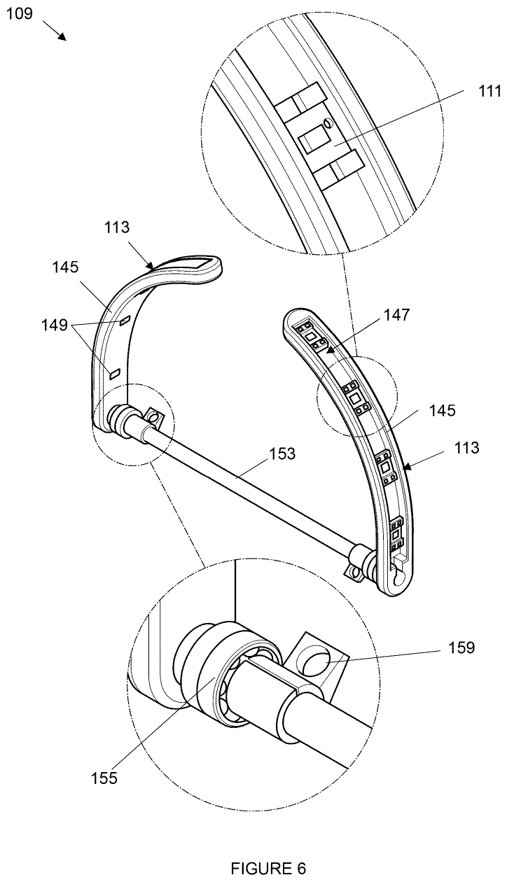

A head circumference measurement assembly (109) is mounted to the base (103) and includes a head circumference measurement means or device configured to measure a head circumference of the infant in a non-contact manner. The head circumference measurement means is provided on a support (113) configured to position the head circumference measurement means at a selected distance from the head of the infant for non-invasive measurement of the head circumference. In the embodiment shown in FIGS. 1 to 6, the support (113) consists of two curved arms (145) and the head circumference measurements means are a plurality of distance sensors (111) mounted to the arms (145). Four distance sensors (111) are equally circumferentially spaced along each curved arm (145) and are enclosed within an internal cavity or chamber (147) defined within each of the curved arms (145), as shown most clearly in FIG. 6. The curved arms (145) include light transmitting and transparent windows (149) for the sensor modules mounted thereto to ensure light passage for distance measurements with the distance sensors (111).

The distance sensors (111) may be ultrasonic, infrared, light emitting diode (LED) time-of-flight or laser sensors. The distance sensors (111) of the embodiment of the apparatus (101) shown in FIGS. 1 to 6 are LED time-of-flight sensors which are relatively small and have higher accuracy and shorter measurement times. It was found that having at least eight distance sensors in a curved sensor array results in suitably accurate head circumference measurements with the minimum amount of interference between the sensors.

The support (113) for the distance sensors (111) is moveable from a stowed condition in which it is at least partially received within a complementarily shaped receiving formation (151) defined in the base (103) and an operative condition in which it extends transversely from the base (103) in a curved or arch-like shape operatively above and partially about the circumference of the infant's head in use. In the embodiment of FIGS. 1 to 6, the receiving formation (151) is a shoulder defined in an outer edge of the base (103) that has a shoulder height which corresponds to the width of the support (113) so that the support (113) lies flush with the operatively upper surface of the base (103) when it is collapsed against the shoulder in the stowed condition. As shown more clearly in FIGS. 4 to 6, the curved arms (145) are pivotally connected to the base (103) via a rotatable shaft (153) that extends through the base when assembled thereto, with the arms mounted to the shaft (153) via ball bearings (155). The rotatable shaft (153) is held captive in the base (103) and ensures that both arms pivot to the same degree relative to the base to convert between the operative and stowed conditions. The support (113) may include reversible locking formations or stabilisation means (157), in this embodiment magnets (159) on the shaft (153), which are configured to lock or stabilise the arms (145) in the operative and stowed (inoperative) conditions, respectively.

Since the support (113) and distance sensors (111) do not contact the infant during measurement and is moved into position for measurement without contacting the infant, the assembly is minimally invasive and the measurement of the head circumference of the infant can be carried out even whilst the infant is receiving constant critical care with medical equipment. The non-contact sensor assembly allows medical personnel or clinicians to measure the head circumference of an infant without removing nCPAP ventilation, for example.

The collapsibility of the length and head circumference measurement assemblies ensures that the apparatus has a generally flat shape allowing it to be slid under an infant for measurements with greater ease and for more compact stowage when not in use.

The weight sensor of the digital scale assembly (105) and distance sensors (211, 229) of the length and head circumference measurement assemblies (107, 109) may be configured to be in communication an onboard processing module in the base (103) or a processing module of an external computer which receives and processes weight, length and head circumference signals from the weight and distance sensors. An onboard processing module may be in communication with a display module (137) fitted to the base which is arranged to display at least the weight, length and head circumference measurements. The weight, length and head circumference measurement data of an infant may be transmitted, preferably wirelessly, to an external computer or mobile device with computer-readable instructions (software) installed thereon for receiving the weight, length and head circumference measured; recording the measurements and storing the measurements in a database.

Accordingly, a computer-implemented method for tracking the growth of an infant is provided comprising obtaining weight, length and head circumference measurements with the anthropometric growth apparatus described herein at specific time intervals (i.e., once a day, or once every second day); storing the measurements in a database associated with the infant; and comparing the measurements with evidence-based growth standards or growth charts. The comparative data may be displayed in a chart. The method may further include using the gestational age of the infant at the time that the measurements are taken to plot weight-for-age, length-for-age and head circumference for age charts and to ensure that the correct growth standards are used. The gestational age of the infant may be specified as an input or may be obtained from further anthropometric measurements carried out with the anthropometric growth apparatus such as the foot length as described in more detail herein below. The measurement obtained with the apparatus may be transmitted to an external computing or mobile device arranged to carry out the further steps of storing and comparing the measurements with evidence-based growth standards or growth charts.

A computer program product for controlling the anthropometric apparatus and tracking the growth of an infant is further provided comprising a computer-readable medium having stored computer-readable program code for performing the steps of recording the weight, length and head circumference measurements made with the anthropometric growth apparatus; and generating plots or graphs of the measurements such as weight-for-age, length-for-age and head circumference-for-age data plots for the infant. The gestational age, sex and demographic factors such as race of the infant may be provided as input for generating the plots. Alternatively, the gestational age may be calculated based on the measurements done with the apparatus. The measurements and plots may be stored in a database associated with an infant. The steps of calibrating the weight and distance sensors and actuating the sensors may also be carried out with the computer and computer program product.

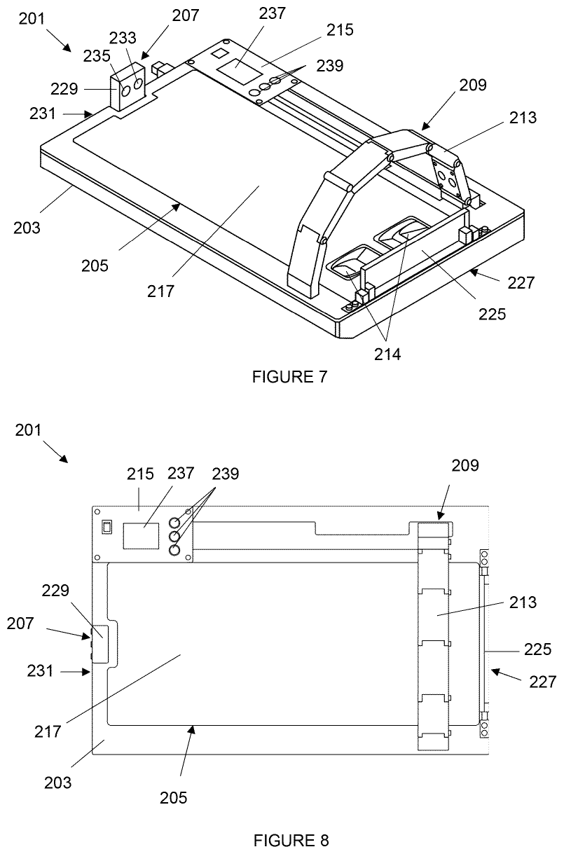

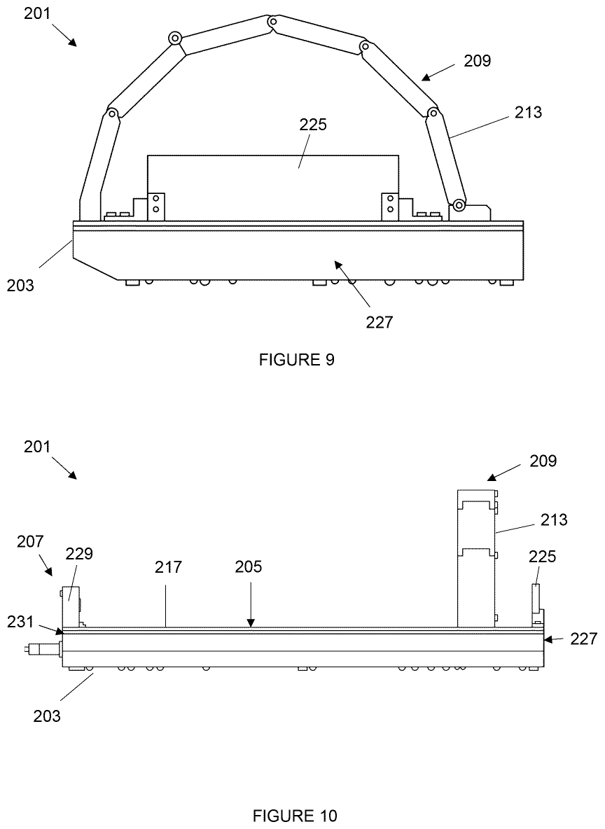

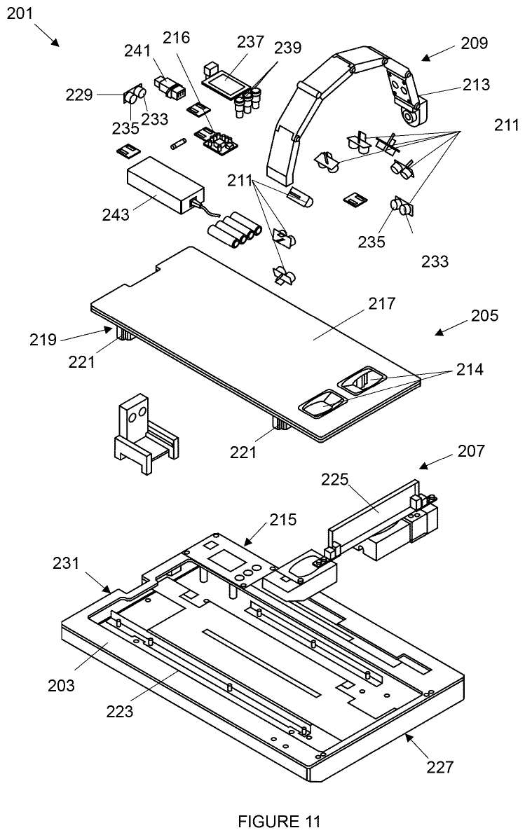

A second embodiment of an anthropometric growth apparatus (201) for measuring an infant is shown in FIGS. 7 to 11 and comprises a base (203), a digital scale assembly (205) which is mounted to the base (203) and configured to measure a weight of the infant and a digital length measurement assembly (207) mounted to the base (203) and configured to measure a length of the infant. A head circumference measurement assembly (209) is also mounted to the base (203) and includes head circumference measurement means configured to measure a head circumference of the infant in a non-contact manner. The head circumference measurement means is provided on a support (213) configured to position the head circumference measurement means at a selected distance from the head of the infant for non-invasive measurement of the head circumference. In the embodiment shown in FIGS. 7 to 11, the head circumference measurement means comprises an array of distance or proximity sensors (211) arranged on an arm or support (213) for the distance sensors (211). The support (213) at least partially extends about and above the circumference of a head of the infant in use to position the array of distance sensors (211) at a selected distance from the head of the infant for non-invasive measurement of the head circumference. The support (213) for the distance sensors (211) may be curved or arc-shaped and extend substantially across the width of the base (203). In the embodiment of FIGS. 7 to 11, the distance sensors (211) are ultrasonic sensors which are robust, easy to calibrate and there is little interference with readings. The mounting angle of an ultrasonic sensor on the support has less of an effect on the head circumference reading obtained in comparison to an IR sensor, for example.

Six of the eight ultrasonic distance sensors (211) are arranged in a semi-circular configuration operatively above an upper or front side of the infant head and a further two distance sensors (211) mounted within the base (203) and below the surface of the base (203) or weighing platform (217) to measure an under- or backside of the head. The apparatus, in particular the weighing platform (217) is provided with transparent windows (214) allowing the distance sensors within the base (203) and below the surface of the platform to transmit and receive light for the distance measurement. The number and arrangement of sensors in the distance sensor array or assembly may of course be optimised depending on the type and specification of sensors used.

The reading cycle of eight ultrasonic sensors is about three seconds to obtain a consistent and stable head circumference measurement. If more ultrasonic sensors are included in the array the overall head circumference accuracy will be improved, but the reading cycle will take longer and any movement of the infant during the reading cycle will affect accuracy.

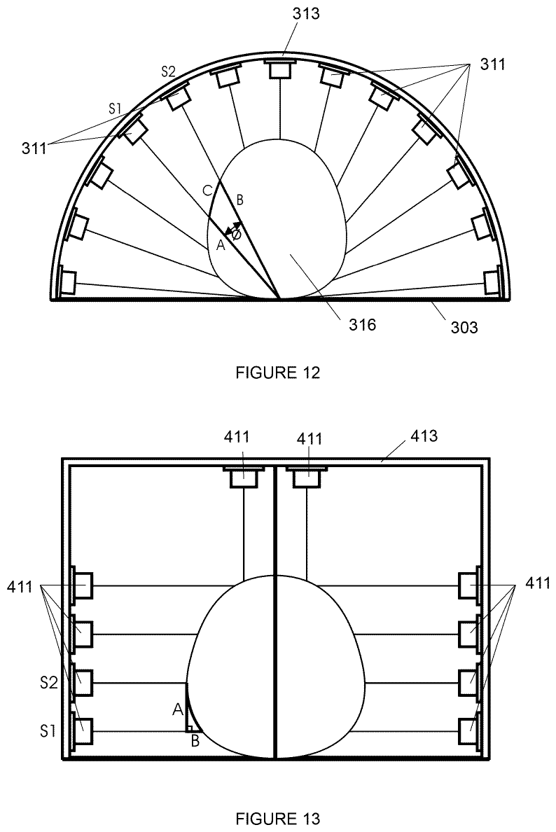

An exemplary array of thirteen distance sensors (311) mounted on a curved, semi-circular shaped support (313) extending across the width of a base (303) or platform is shown in FIG. 12. The Law of Cosines is used to estimate the head circumference by calculating the length of line C in FIG. 12. Distances A and B are calculated by subtracting distance readings S1 and S2 from the distance to the base. The angle Ø is a known and C can then be calculated as follow:

$$C = \sqrt{{A}^{2} + {B}^{2} - 2 AB cos ∅}$$

The curvature of the infant head (316) must be estimated, but the more sensors are used, the more accurate the head circumference measured. For example, more distance sensors can be mounted to a major arc portion of a curved support, i.e., with the sensor support either being entirely circular or with end portions extending beyond a semicircle. The head of the infant may be elevated above the distance sensors arranged at the back of the head for measurement by positioning the head on an upright head support mounted to the base or platform of the apparatus. However, it is preferred that the sensors required to measure a backside of the head be mounted within the base with one or more windows provided in the base or weighing platform, as previously described. In this manner, more sensors can be arranged around the infant head to improve accuracy whilst not requiring invasive positioning of the head on an elevated support.

An alternative sensor array or arrangement is shown in FIG. 13 in which the sensor support (413) is rectangular-shaped and the sensors (411) are linearly arranged on at least three sides (414) of the rectangular-shaped support (413) partially surrounding a head (416) of the infant. The line C in FIG. 13 is calculated using Pythagoras Theorem. Distance A is known due to sensors being mounted in fixed positions on a side of the rectangular-shaped support, and B is calculated by subtracting reading S2 from S1. C is then calculated as follows:

$$C = \sqrt{{A}^{2} + {B}^{2}}$$

Again, the calculation of Line C does not take the curve of the head into consideration. The curve is estimated to get a more accurate circumference measurement and the more sensors used the smaller the curve estimation and the more accurate the total head circumference estimation.

In both abovementioned distance sensor arrangements or arrays, the head circumference is approximated by probing various points on the infant head and applying a spline through the probed points. All the calculations are carried out automatically and the required accuracy is achievable with at least eight sensors in the array.

In the embodiment of FIGS. 7 to 11, the distance sensors (211) are configured to be in communication with a processing module (216) of a control panel (215) which receives and processes head circumference signals from the distance sensors for digital display of a measured head circumference on a control panel (215).

The digital scale assembly (205) of the apparatus (201) of the embodiment of FIGS. 7 to 11 includes a flat and rigid weighing platform (217) on which the infant is placed for weight measurement in use. The platform (217) is provided with a weight sensor (219), in this embodiment multiple force sensors in the form of load cells (221) attached to an underside of the flat platform (217). More particularly, the weight sensor (219) or scale module comprises a flat plate of substantially the same size and shape as the platform (217) with a load cell (221) at each corner. The plate with the four load cells (221) is fixed to an underside of the weighing platform (217). The flat platform (217) and weight sensor (219) are received within a complementarily shaped receiving formation (223), in this embodiment a rectangular-shaped frame, defined in the base (203) so that the load cells are located between the base (203) and the platform (217). The weight sensor (219) is configured to be in communication with a processing module (216) of a control panel (215) which receives and processes a weight signal from the weight sensor for digital display of a measured weight.

The digital length measurement assembly (207) of the embodiment of FIGS. 7 to 11 includes a flat reference board (225) or headboard attached near an end (227) of the base (203) so as to extend at least partially along the width of the base (203) and transversely or generally perpendicular to the base (203) in use to enable a length of the infant to be measured relative to the reference board when a top or bottom extremity of the infant body such as the head or foot of the infant is placed against the reference board in use. A distance sensor (229) is located at or near the opposite end (231) of the base (203) to the reference board (225). The distance sensor (229) is mounted centrally along the width of the base end (231) and is further configured to measure the distance of the infant, in particular a distal end of the infant (head/feet of the infant), from the distance sensor (229). In this embodiment, the distance sensor (229) is an ultrasonic sensor.

The distance sensor (229) is a single ultrasonic sensor with an emitter (233) and receiver (235). The distance sensor (229) is located opposite the headboard (225) or at the foot end of the infant in use. In an alternative embodiment, the digital length measurement assembly may include at least two distance sensors located at or near opposite ends of the elongate base so as to be arranged to measure the distance of the sensors to either end (head and foot end) of the infant or targets placed at the ends of the infant in use in order to calculate the length of the infant. Thus two distance sensors, each with an emitter and receiver, are located at fixed positions at either end of the elongate base and the length of the infant calculated by subtracting the distances measured with the sensors to either end of the infant from the known fixed distance between the two sensors.

To obtain a required length accuracy such as an accuracy within 1 mm, the embodiment of the anthropometric apparatus (201) of FIGS. 7 to 11 includes a high-resolution ultrasonic sensor, such as the M5Stack RCWL-9600 sensor. A temperature probe may be included in the apparatus for ambient temperature compensation since ultrasonic sensors are sensitive to fluctuation in temperature. This may not be necessary when the apparatus is to be used in an incubator or a room with a stable temperature.

The one or more distance sensors of the digital length measurement assembly are configured to be in communication with a processing module of a control panel (215) which receives and processes a length signal from the one or more distance sensors for digital display of a measured length. In other embodiments, the digital length measurement assembly is either a linear digital ruler or a capacitive calliper. The linear digital ruler or capacitive calliper will then be configured to be in communication with the processing module of the control panel (215) which receives and processes the length signal from the linear digital ruler or capacitive calliper for digital display of a measured length.

The embodiment shown in FIGS. 7 to 11 includes a control panel (215) provided with a processing module (216) or controller and a display module (237) or screen. The processing module is in communication with the digital scale, length and head circumference measurement assemblies (205, 207, 209) to receive signals from respective assemblies, process the signals and display at least the weight, length and head circumference measured on the display module (237) which may be an LCD screen for example. The control panel (215) may also include a data input module. In this embodiment, the data input module includes control buttons (239) for recording measurements.

The data input module may also include a keypad for the input of data required to generate weight-for-age, length-for-age and head circumference data plots for the infant. The data required to be input for such plots may be the gender and/or the gestational age at birth of the infant. A software application may be resident on the processing module and executable by the processing module to record the measurements. The processing module may have a clock and a memory or database associated with an infant that stores measurements recorded for the infant at different times. The software application may further be configured to calculate, plot and/or display measurement data, optionally together with the input data from the data input module.

The base (203) is configured to house the electronics and circuitry associated with the sensors of the weight, length and head circumference measurement assemblies (205, 207, 209) and the components of the control panel (215). The embodiment of FIGS. 7 to 11 further includes a connection in the form of a USB port (241) for transferring measurement data or the like to an external computer.

In an alternative configuration in which the apparatus is not provided with a controller or control panel, the apparatus may be provided with connectors or wireless communication means configured to connect or transmit data or signals from the digital scale, length measurement and head circumference measurement assemblies to an external processing module or computer where the data or signals may be processed and the measurements recorded and stored.

The apparatus may be provided with a suitable power supply (243) which may include batteries enabling it to be a portable, point-of care device. The apparatus may of course also be connectable to an external power supply, if required. The apparatus may be configured to be permanently installed in an incubator or the like. The apparatus may have a collapsed, inoperative or stowed condition and an ergonomic design so that it is easily slidable into an incubator and underneath an infant prior to use.

The entire apparatus (201) or various components of the apparatus that may contact the infant in use, such as the platform (217) and reference board (225), for example, may be made of an antimicrobial material or be surface modified and provided with an antimicrobial coating to minimise risk of infection. The apparatus may also be specifically configured and designed to be easily cleaned and sanitised. The platform (217) may be made of a heat-insulating or heat-preserving material to minimise heat loss of the infant during measurement. Preterm infants in particular are unable to regulate their body temperatures and should not be placed on surfaces that conduct heat away from the body.

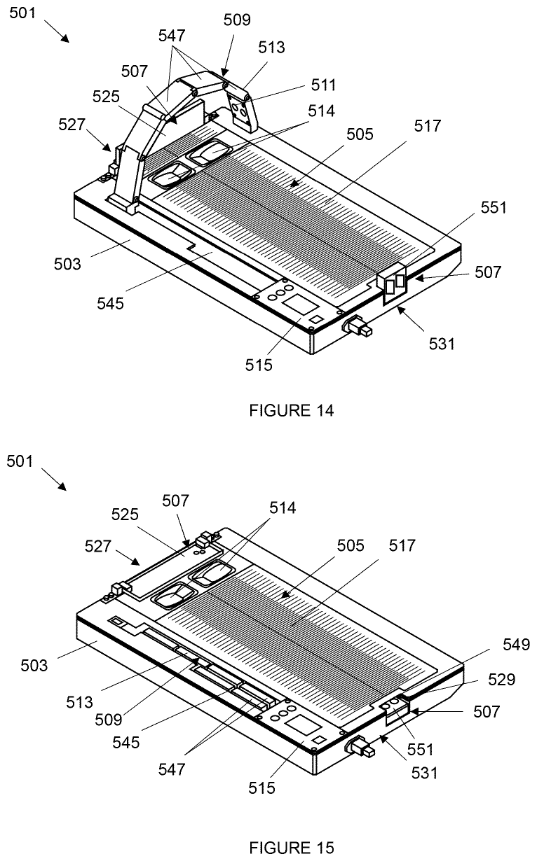

A third embodiment of an anthropometric growth apparatus (501) for measuring an infant is shown in FIGS. 14 to 18. The embodiment is shown in a fully operative, extended condition in FIG. 14 and a stowed or collapsed condition in FIG. 15. The support (513) for the head measurement means may be collapsible enabling the apparatus to be inserted into an incubator with ease and underneath an infant for measurement in a minimally invasive manner. The support (513) for the array of distance sensors (511) may be moveable or collapsible from a stowed or inoperative condition in which it is at least partially contained within a compartment (545) defined in the base (503) and an operative condition in which it is curved and extends partially around the circumference of the infant's head or over the operatively upper portion of the head, i.e, the forehead. The length of the curved support (513) is selected to ensure that the support (513) will not contact the head when it is in the operative condition.

By having a moveable and collapsible support (513) for the sensors, the apparatus (501) can be made compact and thus easy to insert into an incubator or slide underneath an infant prior to measurement. The support (513) is preferably foldable so that it can be stowed in a folded condition in an elongate compartment (545) extending longitudinally along the length of the generally rectangular-shaped base (503) and having a complementary shape to the support (513) in its folded condition. The support (513) may comprise multiple generally rectangular sensor housings (547) or support elements pivotally attached to each other to form a foldable and bendable arm (557). The support may also be pivotally attached to the base so that it can pivot and/or swivel between the stowed and operative conditions. The support (513) with the sensors (511) may be hingedly connected or otherwise mounted to the base (503) near the end (527) of the base provided with a reference headboard (525) or other marking or target indicating where a head of the infant should be positioned prior to measurement of the head circumference. A free end of the support (513) opposite the end mounted to the base (503) may include a magnet arranged to keep the support (513) in its operative position for measurements.

The support (513) of this embodiment is curved in the operative condition. Accordingly, the support arm (513) forms an arc or semicircle spanning substantially across the width of the base (503) near a head end (527) of the base (503). The distance sensors (511) are equally circumferentially spaced along the curved or arc-shaped support arm (513) when in the operative condition. At least six distance sensors (511) are mounted on the curved support arm (513) in separate sensor housings (547). Two additional distance sensors are provided within the base (503) and beneath the upper surface of the base (503) to measure the head circumference at a backside of the infant's head when the infant is lying in a supine position on the apparatus (501) as shown in FIG. 17. Transparent windows (514) are provided within the base (503) of the apparatus (501) for the distance sensors (512) located within the base.

The length measurement assembly (507) of the third embodiment of FIGS. 14 to 18 is moveable between a stowed condition in which the length measurement assembly (507) is collapsed within or against the base (503) and an operative condition in which the assembly extends from the base (503) and is arranged to measure the length of the infant in use. The reference headboard (525) is pivotally attached to the base with a hinge so as to be moveable from the stowed condition in which it is folded against base (503) and/or platform (517) so as to extend generally parallel to the base (503) and platform (517) and an operative condition in which it extends generally perpendicular to the base (503).

The base (503) of this embodiment includes a receptacle (549) defined therein and configured to receive the length measuring distance sensor (529) in the stowed condition. The distance sensor (529) is mounted to the opposite end (531) of the base to the end (527) with the reference board (525). The distance sensor (529) is in a rectangular sensor housing (551) which is slidably and pivotally mounted to the base (503) so as to be slidable and pivotable between the stowed condition in which the distance sensor (529) and sensor housing (551) is at least partially received within the receptacle (549) and the operative condition in which the distance sensor (529) and sensor housing (551) extend transversely or upright from the base.

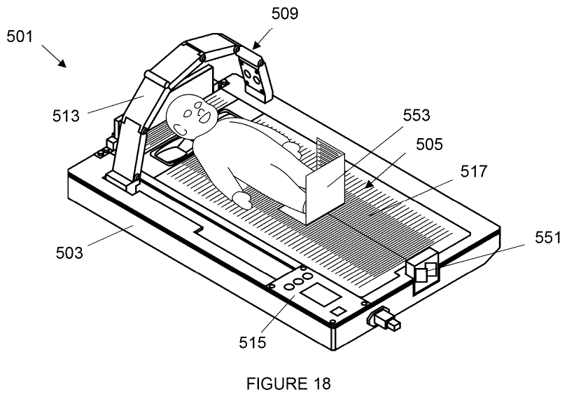

As demonstrated in FIG. 18, a target (553) may be placed at or adjacent the distal end or feet of the infant to measure the distance between the distance sensor (529) and the target (553) with the distance sensor (529) to calculate the length the infant. The target (553) may include a rectangular-shaped board extending transversely from the base (503) or platform (517) of the scale assembly (505) so as to be generally vertical in use. The target may be slidably connected to the base (503) or the like. The target (553) or baseboard placed at the foot end of the infant for length measurements may also be configured to be collapsible, similar to the reference headboard (525) to make the apparatus (501) more compact when not in use. For example, the baseboard or target (553) may be pivotable between a stowed condition in which it extends generally parallel to the base (503) and platform (517) and an operative condition in which it extends transversely from the base (503) or platform (517).

The apparatus may further include gestational age measurement means. There is a strong correlation between the length of an infant's foot and his/her gestational age. The gestational age measurement means may be a digital foot length measurement assembly which may be a linear digital measuring device such as a digital linear tape measure or a capacitive calliper mounted to the base. In one embodiment, the gestational age measurement or estimation means is a camera configured to take images of the infant's foot. The camera may be mounted to the base or may be a standalone camera or a camera associated with a mobile device. The images taken with the camera may be processed by a processing module and the gestational age estimated via a machine learning process involving a convolutional neural network trained to classify images of an infant's foot into different gestational age classes. The infant's demographic factors, gender and weight may influence the gestational age estimate. A specific infant's demographic factors, gender and weight may be passed or input to the neural network to improve its ability to classify the images. To improve the accuracy of the gestational age classification using photographs or images of the foot, the infant's foot may be placed against a transparent plate, such as a transparent plastic plate formed from poly(methyl methacrylate) (PMMA) during imaging. The plate may include binary square fiducial markers (such as Augmented Reality University of Cordoba (ArUco) markers) for use in aligning the plate with the focal plane of the camera. This alignment ensures consistency between the different images. The transparent plate may be attached to the base of the anthropometric growth apparatus via a cord and/or inserted into a slot defined in the base that is configured to house the plate when it is not in use.

The digital foot length measurement assembly may be configured to be in communication with a processing module of the control panel or an external computer or mobile device which receives and processes a foot length signal or images from the camera for digital display of a measured foot length and/or a gestational age estimate.

The target or baseboard to be positioned at or near the foot end of the infant in use for the length measurement may include the digital foot length measurement assembly. The foot length measurement assembly on the target may include a sliding bar that is arranged to move in a vertical direction and may include a variable resistor that changes resistance as it slides. In use, the bar is slid to the tip of the infant toe and the resistance value at the position is read by the processing module and processed or translated into a foot measurement for display on the display module or screen. The foot measurement may be used in a further calculation of an estimated gestational age of the infant.

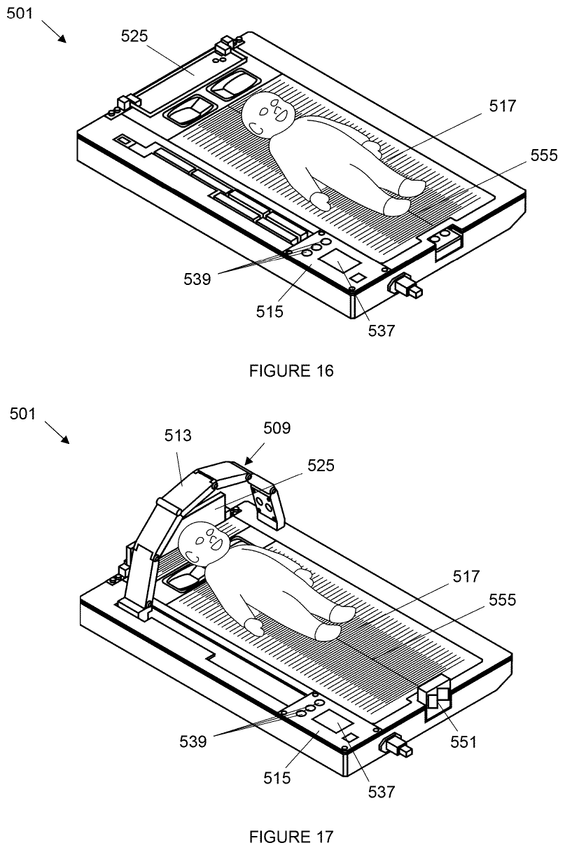

The use of an embodiment of an anthropometric growth apparatus is illustrated in FIGS. 16 to 18. As shown in FIG. 16, the apparatus (501) may still be in the collapsed or stowed condition, yet operative after having been switched on, for weight measurement. Neither the length or head circumference measurement assemblies need to be in their operative positions at this stage. The infant may be placed centrally on the weighing platform (517). Markings (555) may be provided on the platform (517) to assist in positioning the infant appropriately for more accurate readings. The weight measuring function of the control panel (515) or operator interface may be activated with one of the buttons (539) to start measuring and capture the weight of the infant. The captured weight may be displayed on the screen (537).

To measure a length of the infant, the headboard and length sensor housing are moved to their operative conditions. The head circumference sensors do not need to be in position at this stage. Length measurements are taken by placing the infant's head against the generally vertical reference headboard (525) with the legs and feet stretched out towards the sensor in the sensor housing (551). The infant must be centrally placed relative to the width of the base for length measurement. The length measuring function of the control panel (515) may be activated with one of the buttons (539) to measure and capture the length of the infant. The captured length may be displayed on the screen (537). The use of a contactless distance sensor for length measurement is preferred since it enables a single caretaker to complete the measurement since handling of the length measurement assembly itself is not required.

The head circumference measurement assembly (509) may also be moved to its operative condition and securely positioned. The head of the infant should be located within the arch defined by the curved support (513). Appropriate markings may be provided on the base or weighing platform for the correct positioning of the head. The head circumference measuring function of the control panel (515) may be activated with one of the buttons (539) to measure and capture the head circumference of the infant. The captured head circumference may be displayed on the screen (537).

Measurement data capturing, further processing and display using additional data on a specific infant, such as gender or the like, from an input module may also be carried out with the apparatus. Alternatively, measurement or biometric data may be transferred for further processing on an external computer such as a mobile device. A software application resident on the processing module of the apparatus or the external computer may compare the infant growth parameters measured with the apparatus to evidence-based growth standards and growth charts or international prescriptive standards for postnatal growth, such as but not limited to those of the World Health Organisation or the INTERGROWTH-21^{st}, which specifically provides standards for the postnatal growth of preterm infants. This way infant wellbeing can be monitored with the apparatus and nutrition and other prescriptions individualised as needed.

The three most important biometrics to make use of evidence-based growth standards and growth charts such as but not limited to INTERGROWTH-21^{st} standards and tool include: head circumference (accurate to 1 mm); weight (accurate to 10 g) and length/height (accurate to 1 mm).

### Example

### Methods

The accuracy of the apparatus shown in FIGS. 7 to 11 was tested with inanimate objects and following a standard operating procedure described below.

Weight measurement
1. Switch on the apparatus.
2. Calibrate scale on the surface that measurements will take place on.
3. The display will have a zero-weight reading.
4. Place the object to be weighed centrally on the scale platform.
5. Press the “weight button” to activate weight measurement.
6. Reading on the display will start fluctuating.
7. Once the weight reading has stabilized, press the “weight button” to capture the weight.
8. Capture the reading in the test results Excel sheet.
9. Repeat steps 2-6 with 250 g weight 10 times. Moving the weight around on the scale between every reading.
10. Switch off apparatus. Move apparatus around.
11. Repeat steps for 500 g, 1000 g and 2000 g.

### Length Measurement

1. Switch on the apparatus.
2. Open the reference headboard.
3. Slide the ultrasonic length sensor up and out of the base of the unit.
4. Place known 100 mm length object to the headboard on the centre line of the unit.
5. Place target board in front 100 mm object.
6. Press the “length button” to activate the length measurement.
7. Press the “length button” to capture the length.
8. Repeat using 100 mm length measurements×10 times.
9. Switch off apparatus. Move apparatus around.
10. Repeat steps for 250 mm and 500 mm.

### Head Circumference Measurement

1. Switch on the apparatus.
2. Pull the head circumference arm out and attach to the magnets.
3. Press the head circumference button to activate the head circumference measurement.
4. Place the testing instrument with a diameter of 60 mm in the centre.
5. Wait for reading to settle.
6. Press the “head circumference button” to capture the head circumference.
7. Repeat using 60 mm length diameter×10 times.
8. Switch off apparatus. Move apparatus around.
9. Repeat steps for 90 mm and 110 mm.

### Results

### Weight Measurement

The weight was measured at a consistency of 75% (within a 3 g range) at an accuracy of 99.42%, with an average offset of 2.5 g.

TABLE 1
Results of weight measurements.

| | Overall | Average | | Consistency | Consistency | Consistency |
|---|---|---|---|---|---|---|
| Weight | accuracy | reading | Offset | within | within | within |
| (g) | (%) | (g) | (g) | 5 g (%) | 3 g (%) | 1 g (%) |
| 250 | 98.36 | 254.0 | 4.0 | 70 | 70 | 60 |
| 500 | 99.6 | 498.1 | 1.9 | 90 | 80 | 30 |
| 1000 | 99.91 | 999.6 | 0.4 | 100 | 100 | 60 |
| 2000 | 99.82 | 1996.4 | 3.6 | 80 | 50 | 20 |
| | 99.42 | | 2.5 | 85 | 75 | 42.5 |

Factors that may influence the readings obtained include: (i) the platform and the load cells are not secured or connected to each other; (ii) the bed itself has flex, i.e., the material is not rigid enough and (iii) the pressure points on the load cells itself are not the same resulting in inconsistent readings on each single load cell and different measurements due to the placement of the weight on the bed.

### Length Measurement

The length was measured at a consistency of 96.7% (within a 2 mm range) at an accuracy of 99.41%, at an average offset of 0.7 mm.

TABLE 2
Results of length measurements.

| | Overall | Average | | Consistency | Consistency | Consistency |
|---|---|---|---|---|---|---|
| Length | accuracy | reading | Offset | within | within | within |
| (mm) | (%) | (mm) | (mm) | 3 mm (%) | 2 mm (%) | 1 mm (%) |
| 100 | 98.95 | 100.3 | 0.2 | 100 | 100 | 50 |
| 250 | 99.38 | 248.8 | 1.2 | 100 | 90 | 30 |
| 500 | 99.89 | 500.5 | 0.5 | 100 | 100 | 90 |
| | 99.41 | | 0.7 | 100 | 96.7 | 56.7 |

Factors that may influence the readings obtained include: (i) the size of the target—it directly influences reading consistency and is most apparent when the target is small and far away from the sensor; (ii) the levelness of the platform; and (iii) when the moveable sensor is not in the correct position, must be stably seated in the operative condition before measurement.

### Head Circumference Measurement

The circumference was measured at a consistency of 66.7% (within a 3 mm range) at an accuracy of 98.64%, at an average offset of 0.9 mm.

TABLE 3
Results of head circumference measurements.

| | Overall | Average | | Consistency | Consistency | Consistency |
|---|---|---|---|---|---|---|
| Circumference | accuracy | reading | Offset | within | within | within |
| (mm) | (%) | (mm) | (mm) | 5 mm (%) | 3 mm (%) | 1 mm (%) |
| 191 | 97.55 | 191.7 | 0.7 | 80 | 50 | 30 |
| 282 | 98.98 | 280.6 | 1.4 | 80 | 80 | 20 |
| 349 | 99.39 | 349.6 | 0.6 | 100 | 70 | 30 |
| | 98.64 | | 0.9 | 86.7 | 66.7 | 26.7 |

Factors that may influence the readings obtained include: (i) the placement of the object being measured; and (ii) object placement angle.

The apparatus described herein automatically measures multiple parameters quicker and with more ease, thereby decreasing nursing workload and increasing the frequency and accuracy of measurement of infants receiving intensive care in hospital. This will allow timely and appropriate interventions by dietitians and neonatologists to optimise infant growth and development. The apparatus is simple to use and allows for correct measuring and recording of at least three measurement parameters with just one operator. The apparatus may also be used alongside nCPAP and the measurements are obtained in a minimally invasive manner. Preterm infants, i.e., infants born at less than 37 weeks gestation, should not be handled excessively thus requiring minimally invasive measuring procedures. By making the apparatus or parts thereof of antimicrobial materials or designing the apparatus to be easy to clean and sanitize, the apparatus may be safely used for multiple infants in a neonatal ward. The platform may also be made from a heat preserving material to make measurement more comfortable.

The apparatus may be suitably sized and is collapsible so that it can be operated in existing incubators so that infants do not need to be removed from an incubator for anthropometric testing. This decreases the risk for cross contamination and infection. Furthermore, movement and handling of the infant is decreased since the ergonomic, slim line design envisioned does not require the infant to be removed for the incubator and the apparatus can easily be slid under the infant to take measurements. To further minimise handling, the apparatus may also be built into or integrated in incubators.

The apparatus allows for integrated infant measurement data capturing, processing and storage capabilities including software and hardware. As a result, comprehensive growth analysis with growth interpretations provided on standardized growth charts can be done. Any appropriate evidence-based growth standards or growth charts may be employed depending on the age of the infant or child and other characteristics of the infant or child. For example, Fenton Growth Charts or Intergrowth-21 Charts may be used for preterm infants with gestational age less than 37 weeks.

A computer program product for growth tracking and analysis to be used with the apparatus may be in the form of a software application installed on a mobile device. The mobile application, that may be configured to be installed and operable on mobile operating system such as iOS™ and Android™, connects to the anthropometric growth apparatus wirelessly such as via Bluetooth Low Energy (BLE). A full duplex communication protocol involving the concurrent sending and receiving of information allows for system control and data acquisition. Operators or users of the mobile application may collect real time data from the anthropometric growth apparatus, such as head circumference, length and weight measurements. The mobile application may also be used to capture images of the infant's foot from the device's camera to be used for gestational age estimation. Moreover, data collected with the apparatus may be sent by the application to an external server, which stores the collected data in a remote database. This allows for a profile to be created for each infant which can easily be accessed by a doctor at any stage using their own mobile device with the application installed. Additionally, the application may be able to provide control commands to the apparatus including commands to calibrate and tare the scale, for example. The application may also be used to upgrade the anthropometric apparatus firmware via BLE (over the air updates, OTA) removing the need to connect the apparatus to an external computer.

The foregoing description has been presented for the purpose of illustration; it is not intended to be exhaustive or to limit the invention to the precise forms disclosed. Persons skilled in the relevant art can appreciate that many modifications and variations are possible in light of the above disclosure. For example, instead of using distance sensors, the digital length measurement assembly may comprise a linear digital ruler. The linear digital ruler may extend longitudinally to the elongate base to measure the length of the infant relative to the reference headboard in use. In a further alternative embodiment, the length measurement assembly may be a collapsible capacitive calliper mounted to the base. Linear digital measurement devices include two variable, out-of-phase capacitors. The capacitance changes linearly as the grid capacitors slide over each other while a signal processing unit counts the grids as the slider moves and, due to the linear change in capacitance, performs linear interpolation. This gives the device the exact position of the slider. Devices can be designed to have an accuracy of under 1 mm. Similar to the length sensor assembly, the linear digital ruler or collapsible capacitive calliper may be configured to be in communication with a processing module which receives and processes a length signal from the linear digital ruler or capacitive calliper for digital display of a measured length.

Any suitable head positioning means or marking, not necessarily a reference board, may be included as part of the length measurement assembly in the apparatus.

Another potential non-contact method of measuring body length and foot length is three-dimensional scanning. The apparatus may thus include a 3D scanner as part of the digital length measurement assembly or the foot measurement assembly. The scans are saved as three-dimensional virtual objects which allow for the biometrics to be measured using Computer-Aided Design (CAD) software for example. The required measurement accuracy is achievable with 3D scanners, but it makes the apparatus more expensive and thus less suitable for a limited resource setting.

The platform of the digital scale assembly may be any suitable size and may be sized and configured so that it is fully separate from the head circumference measurement assembly. The digital scale assembly may include a permanent scale platform which includes a stable weighing platform with a weight sensor comprising force transducers or sensors attached thereto as herein described before or a removeable scale module may be used. This configuration may be useful when the apparatus is configured to be permanently installed in an incubator. Rather than having a scale module including a weight sensor permanently mounted to the apparatus, a removeable scale module may be used to take measurements after sliding the module underneath a stable mattress platform. The mattress platform would then be mechanically lowered onto the scale module for weight measurement. The apparatus may include a mechanical cam lifting system, such as a cam lifting arm, arranged to lower the platform and infant onto the scale module when the scale module is in position on the apparatus. Measurement signals and data may be captured as previously described.

The apparatus described herein automatically and conveniently provides combined measurement data which may include, but are not limited to infant weight, height, foot length and head circumference. Further measurement assemblies such as body temperature sensors or the like may also be incorporated in the apparatus. The apparatus may also include body composition measurement means capable of determining muscle and/or fat mass in the infant body. Any suitable sensors including ultrasound or ultrasonic sensors and probes and/or bioelectrical impedance measurement analyzers may be used for body composition determination. Alternatively, the head circumference measurement means or device and its support may be moveable along the length of the base or platform and be adapted to measure the circumference of the infant body at various different positions along the length of the body. These measurements may be used to calculate specific body part circumference ratios for estimating body composition. Body composition measurements are also important for tracking the development of the infant. Measurements of body part circumferences including but not limited to head circumference and mid-upper arm circumference, together with the weight and length of the infant, may also be used to estimate the gestational age of the infant.

The gestational age of the infant may also be estimated using a combination of as many as possible of the following measurements: weight, head circumference, mid-upper arm circumference, crown heel length, crown rump length, foot length and hand length. It will be appreciated that the crown rump length may be measured with the length measurement assembly of the apparatus described herein by lifting the legs of the infant to extend generally perpendicularly to the base or weighing platform when the infant is lying down on the platform. The foot length and hand length may be measured with digital calipers, a digital ruler or digital tape measure mounted to the base or by taking images of the hands and feet with a camera, optionally mounted to the base, and using the image processing and machine learning classification technique described herein.

It will be appreciated that the size of the apparatus may be adapted so that it is configured to be used in pediatric anthropometry to measure the anthropometry of preterm neonates, neonates, infants, toddlers or even children up to the age of about 13 years. In the case of toddlers or older children, the apparatus may find use in obtaining anthropometric measurements of disabled, injured or otherwise fragile children that are unable to stand upright for body measurements.

The language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

Finally, throughout the specification and accompanying claims, unless the context requires otherwise, the word ‘comprise’ or variations such as ‘comprises’ or ‘comprising’ will be understood to imply the inclusion of a stated integer or group of integers but not the exclusion of any other integer or group of integers.

1. An anthropometric growth apparatus for measuring the anthropometry of an infant comprising:
a base;
a digital scale assembly mounted to the base and configured to measure a weight of the infant;
a digital length measurement assembly mounted to the base and configured to measure a length of the infant; and
a head circumference measurement assembly mounted to the base which includes an array of distance sensors provided on a support configured to position the array of distance sensors at a selected distance from a head of the infant for non-invasive measurement of a head circumference of the infant in use.

2. The anthropometric growth apparatus as claimed in claim 1, wherein the support for the array of distance sensors is moveable from a stowed condition in which it is at least partially received within a complementarily shaped receiving formation defined in the base and an operative condition in which it extends transversely from the base so as to be operatively above and extend at least partially around the circumference of the infant's head in use.

3. The anthropometric growth apparatus as claimed in claim 2, wherein the support includes at least one curved arm with the distance sensors equally circumferentially spaced along the curved arm.

4. The anthropometric growth apparatus as claimed in claim 1, wherein the support is pivotally attached to the base so as to be collapsible against the base.

5. The anthropometric growth apparatus as claimed in claim 1, wherein the digital length measurement assembly includes a reference board attached at or near an end of the base and extending transversely therefrom in use and a distance sensor at or near the opposite end of the base, the distance sensor being configured to measure the distance of the infant, or a target placed at or adjacent the infant, from the distance sensor for use in calculating a length of the infant.

6. The anthropometric growth apparatus as claimed in claim 1, wherein the digital length measurement assembly is moveable between a stowed condition in which the length measurement assembly is collapsed within or against the base and an operative condition in which the assembly extends transversely from the base and is arranged to measure the length of the infant in use.

7. The anthropometric growth apparatus as claimed in claim 5, wherein the reference board and distance sensor of the digital length measurement assembly are pivotally attached to the base so as to be moveable between the stowed condition and the operative condition.

8. The anthropometric growth apparatus as claimed in claim 5, wherein the base has recesses defined therein and configured to receive the reference board and distance sensor in the stowed condition.

9. The anthropometric growth apparatus as claimed in claim 1, wherein the digital scale assembly includes a flat, weighing platform on which the infant is placed for weight measurement in use with a weight sensor on the platform.

10. The anthropometric growth apparatus as claimed in claim 1, further including a digital foot length measurement assembly.

11. The anthropometric growth apparatus as claimed in claim 1, wherein the apparatus includes a processing module in communication with the digital weight, length and head circumference measurement assemblies to receive signals from the respective assemblies and process the signals to determine the weight, length and head circumference of the infant.

12. The anthropometric growth apparatus as claimed in claim 11, wherein the apparatus includes a display module on the base that is in communication with the processing module and configured to display the weight, length and head circumference of the infant in use.

13. The anthropometric growth apparatus as claimed in claim 11, wherein the processing module is configured to transmit the measurements to a processing module of an external computing device for further processing.
